(19) United States
(12) Patent Application Publication
(10) Pub. No.: US 2021/0346269 A1
(43) Pub. Date: Nov. 11, 2021

(54) ACTIVE SUBSTANCE COMPOSITION FOR MODIFYING HAIR SURFACE AND CHANGING THE SHAPE OF HAIR

(71) Applicant: Henkel AG & Co. KGaA, Duesseldorf (DE)

(72) Inventors: RENE KROHN, Norderstedt (DE); ERIK SCHULZE ZUR WIESCHE, Bielefeld (DE); TORSTEN LECHNER, Langenfeld (DE)

(73) Assignee: Henkel AG & Co. KGaA, Duesseldorf (DE)

(21) Appl. No.: 17/288,873

(22) PCT Filed: Oct. 31, 2019
(86) PCT No.: PCT/EP2019/079768

§ 371 Date: Apr. 26, 2021

(30) Foreign Application Priority Data
Oct. 31, 2018 (DE) 10 2018 127 225.1

(51) Int. Cl. A61K 8/58; A61K 8/81; A61K 8/85; A61Q 5/06; A61Q 5/12

(52) U.S. Cl. CPC A61K 8/585; A61K 8/8147; A61K 8/8182; A61K 2800/5922; A61Q 5/06; A61Q 5/12; A61K 8/85

(57) ABSTRACT

A cosmetic agent for treating a keratinous material contains a) at least one organic silicon compound and b) at least one polycarboxylic acid, wherein the cosmetic agent is particularly suitable for providing structural thermal protection.

### CROSS-REFERENCE TO RELATED APPLICATION

This application is a U.S. National-Stage entry under 35 U.S.C. § 371 based on International Application No. PCT/EP2019/079768, filed Oct. 31, 2019, which was published under PCT Article 21(2) and which claims priority to German Application No. 10 2018 127 225.1, filed Oct. 31, 2018, which are all hereby incorporated in their entirety by reference.

### TECHNICAL FIELD

The present disclosure relates to cosmetic agents for treating a keratinous material, the agent comprising an organic silicon compound and at least one polycarboxylic acid, and the use of the cosmetic agent.

### BACKGROUND

The external exposure of hair to chemicals from a variety of different sources poses challenges for the development of cosmetic products. Air and water impurities have a detrimental effect on skin and hair. The most important air pollutants include polycyclic aromatic hydrocarbons, volatile organic compounds, nitrogen oxides (NOx), particles and cigarette smoke. The effect of various air pollutants can be enhanced in the presence of other air pollutants and when exposed to UV radiation or heat.

It is known that the toxicity of gaseous pollutants of air, such as sulfur dioxide, ozone and nitrogen oxides, is related in particular to their initiator activity for free radicals which cause damage to living organisms. Free radicals are metabolites that also occur naturally in the body. In large quantities, free radicals can promote irritation and inflammation and accelerate the process of aging. In this case, one speaks of “oxidative damage” Free radicals can also cause visible hair damage, for example, as a reduction in shine as well as grip and/or fading of hair color. Heat exposure naturally increases the damage caused by pollutants.

However, customer requests do not only concern characteristics that affect the appearance of the hair itself. The profile of requirements for cosmetic agents for hair is more diverse. Cosmetic agents that serve a permanent or temporary shaping of the hair also play an important role in cosmetics. Thus, cosmetic agents should provide temporary shaping with good hairstyle retention. The development of cosmetic agents that enable good hair hold and provide a protective function against damage from pollutants is a challenge.

In the prior art, organosilicon compounds from the group of silanes comprising at least one hydroxy group and/or hydrolyzable group are described. Due to the presence of hydroxy groups and/or hydrolyzable groups, silanes are reactive substances that hydrolyze or oligomerize or polymerize in the presence of water. The oligomerization or polymerization of the silanes initiated by the presence of the water, when applied to a keratinous material, ultimately leads to the formation of a film that can provide a protective effect.

### BRIEF SUMMARY

A cosmetic agent for the treatment of a keratinous material contains

a) at least one organic silicon compound and

b) at least one polycarboxylic acid composed of at least one first monomer of the formula (A)

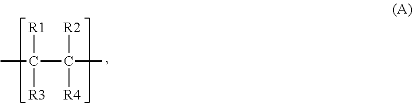

wherein

R1 and R3 independently of one another represent H, CH₃, OH, (CH₂)ₙOH, NH₂, (CH₂)NH₂, COOH, (CH₂)ₙCOOH, COOX, (CH₂)ₙCOOX, in particular H, and n is from 0 to 6 or from 1 to 6,

R2 represents H, CH₃, OH, (CH₂)nOH, NH₂, (CH₂)NH₂, COOH, (CH₂)ₙCOOH, COOX, (CH₂)ₙCOOX with n equal to from 0 to 6 or from 1 to 6, in particular H, CH₃, COOH or COOX, and

R4 represents (CH₂)ₙCOOH or (CH₂)nCOOX with n equal to 0 to 6 or from 1 to 6, in particular 1,

wherein X in R1, R2, R3, R4 represents an alkali or alkaline earth metal ion or an ammonium ion.

In various embodiments, the polycarboxylic acid is a homopolymer composed of itaconic acid or a salt of itaconic acid, or the polycarboxylic acid is a copolymer of formula (B):

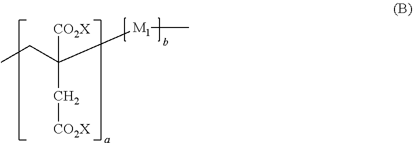

In formula (B), M₁ represents one or more different repeating units representing one or more different vinyl monomer units; X represents hydrogen or an alkali metal or alkaline earth metal, a is the molar fraction of the itaconic acid-derived repeating unit in the copolymer, and b is the molar fraction of the one or more different repeating units M₁ in the copolymer, where a is from about 20 mol % to less than 100 mol %, preferably from 50 mol % to less than 100 mol %, and b is from more than 0 mol % to 50 mol %, preferably from more than 0 mol % to 80 mol %.

In various embodiments, the at least one organic silicon compound comprises a compound of the formula (I) or of formula (II),
wherein in the organic silicon compound of formula (I)

R₁R₂N-L-Si(OR₃)ₐ(R₄)_{b} (I),
R₁, R₂ both represent a hydrogen atom,
L represents a linear, divalent C₁-C₆-alkylene group, preferably a propylene group (—CH₂—CH₂—CH₂—) or an ethylene group (—CH₂—CH₂—),
R₃, R₄ independently represent a methyl group or an ethyl group,
a stands for the number 3 and
b stands for the number 0, and
wherein in the organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
R₅, R_{5′}, R_{5″}, R₆, R_{6′} and R_{6″} stand independently for a C₁-C₆ alkyl group,
A, A′, A″, A″′ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

(A″″)-Si(R₆″)_{d}″(OR₅″)_{c}″ (III),
c stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
with the proviso that at least one of the components of e, f, g and h is different from 0.

Suitable organic silicon compounds of formula (I) include
(3-Aminopropyl)trimethoxysilane
(3-Aminopropyl)triethoxysilane
(2-Aminoethyl)trimethoxysilane
(2-Aminoethyl)triethoxysilane
(3-Dimethylaminopropyl)trimethoxysilane
(3-Dimethylaminopropyl)triethoxysilane
(2-dimethylaminoethyl)trimethoxysilane, and
(2-Dimethylaminoethyl)triethoxysilane.

Suitable organic silicon compounds of formula (II) include

3-(trimethoxysilyl)-N-[3-(trimethoxysilyl)propyl]-1-propanamine

3-(Triethoxysilyl)-N-[3-(triethoxysilyl)propyl]-1-propanamine

N-methyl-3-(trimethoxysilyl)-N-[3-(trimethoxysilyl)propyl]-1-propanamine

N-Methyl-3-(triethoxysilyl)-N-[3-(triethoxysilyl)propyl]-1-propanamine

2-[Bis[3-(trimethoxysilyl)propyl]amino]-ethanol
2-[Bis[3-(triethoxysilyl)propyl]amino]ethanol

3-(Trimethoxysilyl)-N,N-bis[3-(trimethoxysilyl)propyl]-1-propanamine

3-(Triethoxysilyl)-N,N-bis[3-(triethoxysilyl)propyl]-1-propanamine

N1,N1-bis[3-(trimethoxysilyl)propyl]-1,2-ethanediamine,

N1,N1-bis[3-(triethoxysilyl)propyl]-1,2-ethanediamine,

N,N-Bis[3-(trimethoxysilyl)propyl]-2-propen-1-amine and

N,N-Bis[3-(triethoxysilyl)propyl]-2-propen-1-amine.

In further embodiments, the organic silicon compound is selected from those of formula (IV),

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

Suitable compounds are selected from the group consisting of

Methyltrimethoxysilane;

Methyltriethoxysilane;

Ethyltrimethoxysilane;

Ethyltriethoxysilane;

Propyltrimethoxysilane;

Propyltriethoxysilane;

Hexyltrimethoxysilane;

Hexyltriethoxysilane;

Octyltrimethoxysilane;

Octyltriethoxysilane;

Dodecyltrimethoxysilane;

Dodecyltriethoxysilane;

Octadecyltrimethoxysilane; and

Octadecyltriethoxysilane.

Methods of treating keratinous material involve applying a cosmetic agent containing these silicon compounds and the polycarboxylic acid onto the keratinous material. In one embodiment, the method involves applying an aqueous solution comprising the organic silicon compound onto the keratinous material and applying an aqueous solution comprising the polycarboxylic acid onto the keratinous material.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The underlying problem of the present disclosure is to provide a cosmetic product that allows good shaping of hair and improves structural protection against the effects of heat.

This problem is solved by a cosmetic agent for the treatment of a keratinous material, comprising
a) at least one organic silicon compound and
b) at least one polycarboxylic acid which is composed of at least one first monomer of the formula (A)

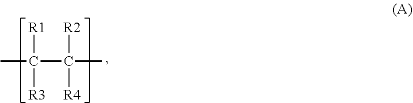

wherein
R1 and R3 independently of one another represent H, CH₃, OH, (CH₂)ₙOH, NH₂, (CH₂)NH₂, COOH, (CH₂)ₙCOOH, COOX, (CH₂)ₙCOOX, in particular H, and n is from 0 to 6 or from 1 to 6,
R2 represents H, CH₃, OH, (CH₂)nOH, NH₂, (CH₂)NH₂, COOH, (CH₂)ₙCOOH, COOX, (CH₂)ₙCOOX with n equal to 0 to 6 or 1 to 6, in particular H, CH₃, COOH or COOX, and
R4 represents (CH₂)ₙCOOH or (CH₂)nCOOX with n equal to 0 to 6 or 1 to 6, in particular 1,
wherein X in R1, R2, R3, R4 represents an alkali or alkaline earth metal ion or an ammonium ion.

Keratinous material includes hair, skin, nails (such as fingernails and/or toenails). Wool, furs and feathers also fall under the definition of keratinous material.

Preferably, keratinous material is understood to be human hair, human skin and human nails, especially fingernails and toenails. In particular, keratinous material is understood to mean human hair, especially head and/or beard hair.

As a first ingredient essential to the invention, the cosmetic agent for treating a keratinous material contains at least one organic silicon compound. Preferred organic silicon compounds are selected from silanes having one, two or three silicon atoms, wherein the organic silicon compound comprises one or more hydroxyl groups and/or hydrolyzable groups per molecule.

Organic silicon compounds, alternatively called organosilicon compounds, are compounds which either have a direct silicon-carbon bond (Si—C) or in which the carbon is bonded to the silicon atom via an oxygen, nitrogen or sulfur atom. The organic silicon compounds are compounds containing one to three silicon atoms. Organic silicon compounds preferably contain one or two silicon atoms.

According to IUPAC rules, the term silane stands for a group of chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

The agent for treating a keratinous material contains at least one organic silicon compound preferably selected from silanes having one, two or three silicon atoms, wherein the organic silicon compound comprises one or more hydroxyl groups or hydrolyzable groups per molecule.

In a particularly preferred embodiment, the agent for treating a keratinous material comprises at least one organic silicon compound selected from silanes having one, two or three silicon atoms, the organic silicon compound further comprising one or more basic groups and one or more hydroxyl groups or hydrolyzable groups per molecule.

This basic group can be, for example, an amino group, an alkylamino group or a dialkylamino group, which is preferably connected to a silicon atom via a linker. The basic group is preferably an amino group, a C₁-C₆ alkylamino group or a Di(C₁-C₆)alkylamino group.

The hydrolyzable group(s) is (are) preferably a C₁-C₆ alkoxy group, especially an ethoxy group or a methoxy group. It is preferred when the hydrolyzable group is directly bonded to the silicon atom. For example, if the hydrolyzable group is an ethoxy group, the organic silicon compound preferably contains a structural unit R′R″R″′Si—O—CH₂—CH₃. The residues R′, R″ and R″′ represent the three remaining free valences of the silicon atom.

Particularly good results were obtained when the agent for treating a keratinous material contains at least one organic silicon compound of formula (I) and/or (II).

The compounds of formulae (I) and (II) are organic silicon compounds selected from silanes having one, two or three silicon atoms, the organic silicon compound comprising one or more hydroxyl groups and/or hydrolysable groups per molecule.

In another very particularly preferred embodiment, the agent for treating a keratinous material comprises at least one organic silicon compound of formula (I) and/or (II),

R₁R₂N-L-Si(OR₃)ₐ(R₄)_{b} (I),
where

R₁, R₂ both represent a hydrogen atom, and

L represents a linear, divalent C₁-C₆-alkylene group, preferably a propylene group (—CH₂—CH₂—CH₂—) or an ethylene group (—CH₂—CH₂—),

R₃, R₄ independently represent a methyl group or an ethyl group, and

a stands for the number 3 and
b stands for the number 0.

(R₅O)_{c}(R₆)_{d}Si-(A)ₑ-[NR₇-(A′)]_{f}-[O-(A″)]_{g}[NR₈-(A′″)]ₕ-Si(R₆′)_{d′}(OR₅′)_{e′} (II),

where
R5, R5′, R5″ independently represent a hydrogen atom or a C₁-C₆ alkyl group,
R6, R6′ and R6″ independently represent a C₁-C₆ alkyl group,

A, A′, A″, A″′ and A″″ independently represent a linear or branched divalent C₁-C₂₀ alkylene group,

R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

(A″″)-Si(R₆″)_{d}″(OR₅″)_{c}″ (III),

c, stands for an integer from 1 to 3,

d stands for the integer 3-c,

c′ stands for an integer from 1 to 3,

d′ stands for the integer 3-c′,

c″ stands for an integer from 1 to 3,

d″ stands for the integer 3-c″,

e stands for 0 or 1,

f stands for 0 or 1,

g stands for 0 or 1,

h stands for 0 or 1,

provided that at least one of e, f, g and h is different from 0.

The substituents R₁, R₂, R₃, R₄, R₅, R₅′, R₅″, R₆, R₆′, R₆″, R₇, R₈, L, A′, A″, A′″ and A″″ in the compounds of formula (I) and (II) are explained below as examples: Examples of a C₁-C₆ alkyl group are the groups methyl, ethyl, propyl, isopropyl, n-butyl, s-butyl and t-butyl, n-pentyl and n-hexyl. Propyl, ethyl and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear divalent C₁-C₂₀ alkylene group include the methylene group (—CH₂—), the ethylene group (—CH₂—CH₂—), the propylene group (—CH₂—CH₂—CH₂—) and the butylene group (—CH₂—CH₂—CH₂—). The propylene group (—CH₂—CH₂—CH₂—) is particularly preferred. From a chain length of 3 C atoms, divalent alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L-, which stands for a linear or branched, divalent C₁-C₂₀ alkylene group.

Preferably, -L- represents a linear, divalent C₁-C₂₀ alkylene group. More preferably -L- stands for a linear divalent C₁-C₆ alkylene group. Particularly preferred -L stands for a methylene group (—CH₂—), an ethylene group (—CH₂—CH₂—), propylene group (—CH₂—CH₂—CH₂—) or butylene (—CH₂—CH₂—CH₂—CH₂—). In particular, L stands for a propylene group (—CH₂—CH₂—CH₂—)

The organic silicon compounds of formula (I)

R₁R₂N-L-Si(OR₃)ₐ(R₄)_{b} (I),

one end of each carries the silicon-containing group —Si(OR₃)ₐ(R₄)_{b}

In the terminal structural unit —Si(OR₃)ₐ(R₄)_{b}, R₃ is hydrogen or C₁-C₆ alkyl group, and R₄ is C₁-C₆ alkyl group. In particular, R₃ and R₄ independently of each other represent a methyl group or an ethyl group.

Here a stands for an integer from about 1 to about 3, and b stands for the integer 3-a. If a stands for the number 3, then b is equal to 0. If a stands for the number 2, then b is equal to 1. If a stands for the number 1, then b is equal to 2.

The best protection against the negative effects of water and/or air pollution (“anti-pollution” effect) and the best care of stressed hair could be obtained if the agent for treating a keratinous material contains at least one organic silicon compound of formula (I) in which the radicals R₃, R₄ independently represent a methyl group or an ethyl group.

Particularly well-suited organic silicon compounds of formula (I) are

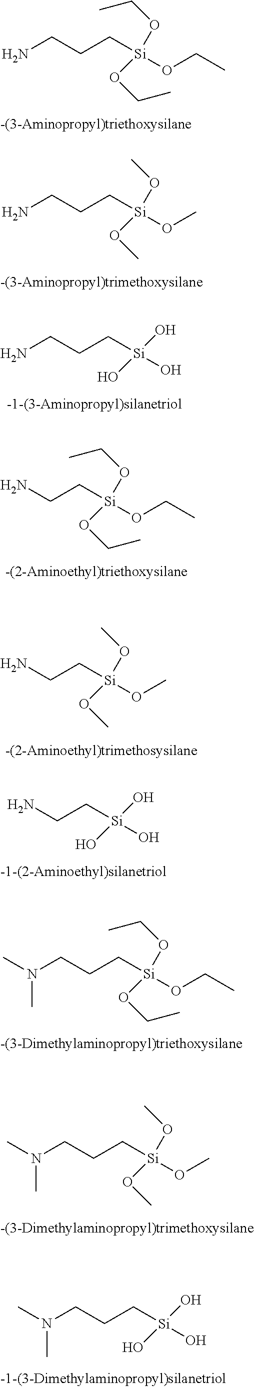

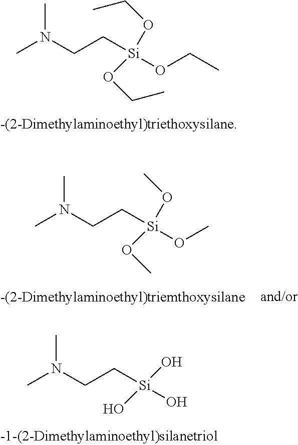

The aforementioned organic silicon compound of formula (I) is commercially available.

(3-aminopropyl)trimethoxysilane, for example, can be purchased from Sigma-Aldrich. (3-Aminopropyl)triethoxysilane is also commercially available from Sigma-Aldrich.

In another embodiment, the composition for treating a keratinous material comprises at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

The organosilicon compounds of formula (II) [MF1] each carry the silicon-containing groups (R₅O)_{c}(R₆)_{d}Si— and —Si(R₆′)_{d′}(OR₅′)_{c} at both ends.

In the central part of the molecule of formula (II) there are the groups -(A)ₑ- and —[NR₇-(A′)]_{f}- and —[O-(A″)]_{g}- and —[NR₈-(A′″)]ₕ-. Here, each of the radicals e, f, g and h can independently of one another stand for the number 0 or 1, with the proviso that at least one of the radicals e, f, g and h is different from 0. In other words, an organic silicon compound of formula (II) contains at least one grouping from the group consisting of -(A)- and —[NR₇-(A′)]- and —[O-(A″)]- and —[NR₈-(A′″)]-.

In the two terminal structural units (R₅O)_{c}(R₆)_{d}Sii- and —Si(R₆′)_{d′}(OR₅′)_{c}, the radicals R5, R5′, R5″ independently of one another represent a hydrogen atom or a C₁-C₆ alkyl group. The radicals R6, R6′ and R6″ independently represent a C₁-C₆ alkyl group.

Here a stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

A very high anti-pollution effect of the agent for the treatment of a keratin material could be obtained if the residues c and c′ both represent the number 3. In this case d and d′ both stand for the number 0.

In a further preferred embodiment, the agent for treating a keratin material contains at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
R5 and R5′ independently represent a methyl group or an ethyl group,
c and c′ both stand for the number 3 and
d and d′ both stand for the number 0.

If c and c′ are both the number 3 and d and d′ are both the number 0, the organic silicon compound corresponds to formula (IIa)

(R₅O)₃Si-(A)ₑ-[NR₇-(A′)]_{f}-[O-(A″)]_{g}-[NR₈-(A′″)]ₕ-Si(OR₅′)₃ (IIa).

The radicals e, f, g and h can independently stand for the number 0 or 1, whereby at least one radical from e, f, g and h is different from zero. The abbreviations e, f, g and h thus define which of the groupings -(A)ₑ- and —[NR7-(A′)]_{f}- and —[O-(A″)]_{g}- and —[NR8-(A′″)]ₕ- are located in the middle part of the organic silicon compound of formula (II).

In this context, the presence of certain groups has proved to be particularly advantageous in terms of increasing the “anti-pollution” effect. Particularly good results were obtained when at least two of the residues e, f, g and h stand for the number 1. Especially preferred e and f both stand for the number 1. Furthermore, g and h both stand for the number 0.

If e and f both stand for the number 1 and g and h both stand for the number 0, the organic silicon compound as contemplated herein corresponds to formula (IIb)

(R₅O)_{c}(R₆)_{d}Si-(A)-[NR₇-(A′)]-Si(R₆′)_{d′}(OR₅′)_{c′} (IIb).

The radicals A, A′, A″, A″′ and A″″ independently represent a linear or branched divalent C₁-C₂₀ alkylene group. Preferably the radicals A, A′, A″, A″′ and A″″ independently of one another represent a linear, divalent C₁-C₂₀ alkylene group. Further preferably the radicals A, A′, A″, A′″ and A″″ independently represent a linear divalent C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A′″ and A″″ stand for a propylene group (—CH₂—CH₂—CH₂—).

When the radical f represents the number 1, the organic silicon compound of formula (II) contains a structural grouping —[NR7-(A′)]-.

When the radical h represents the number 1, the organic silicon compound of formula (II) contains a structural grouping —[NR8-(A″)]-.

Here R₇ and R₈ independently stand for a hydrogen atom, a C₁-C₆ alkyl group, a hydroxy-C₁-C₆ alkyl group, a C₂-C₆ alkenyl group, an amino-C₁-C₆ alkyl group or a group of the formula (III)

-(A″″)-Si(R₆″)_{d}″(OR₅″)_{c}″ (III),

Very preferably, R₇ and R₈ independently stand for a hydrogen atom, a methyl group, a 2-hydroxyethyl group, a 2-alkenyl group, a 2-aminoethyl group or a group of formula (III).

If the radical f represents the number 1 and the radical h represents the number 0, the organic silicon compound contains the grouping [NR₇-(A′)] but not the grouping —[NR₈-(A′″)]. If the residue R7 is a grouping of formula (III), the agent for treating a keratin material contains an organic silicon compound with 3 reactive silane groups.

In a further preferred embodiment, the agent for treating a keratin material contains at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
e and f both stand for the number 1,
g and h both stand for the number 0,
A and A′ independently of one another represent a linear, divalent C₁-C₆ alkylene group and
R7 represents a hydrogen atom, a methyl group, a 2-hydroxyethyl group, a 2-alkenyl group, a 2-aminoethyl group or a group of formula (III).

In another preferred embodiment, the agent for treating a keratin material contains at least one organic silicon compound of formula (II), wherein
e and f both stand for the number 1,
g and h both stand for the number 0,
A and A′ independently of one another represent a methylene group (—CH₂—), an ethylene group (—CH₂—CH₂—) or a propylene group (—CH₂—CH₂—CH₂),
   and
R₇ represents a hydrogen atom, a methyl group, a 2-hydroxyethyl group, a 2-alkenyl group, a 2-aminoethyl group or a group of formula (III).

Organic silicon compounds of formula (II) that are suitable for solving the problem are

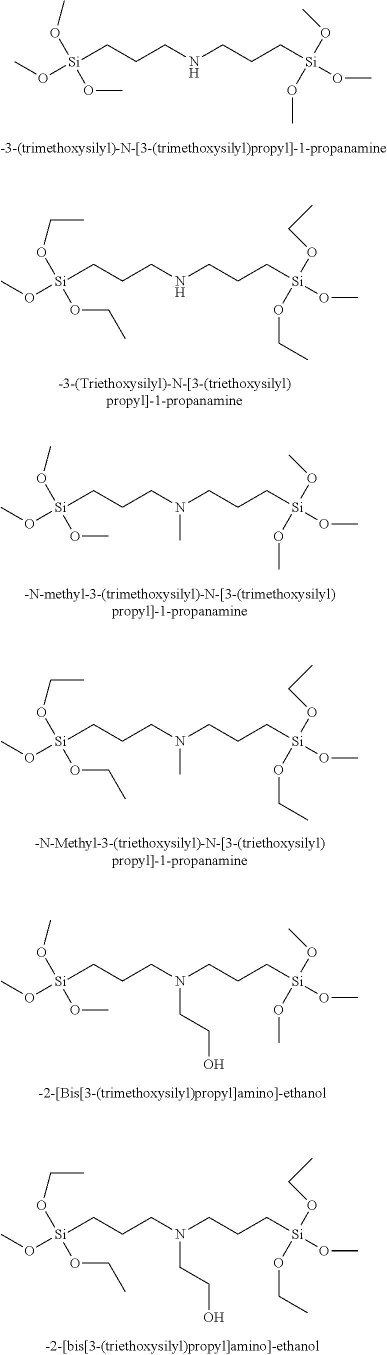

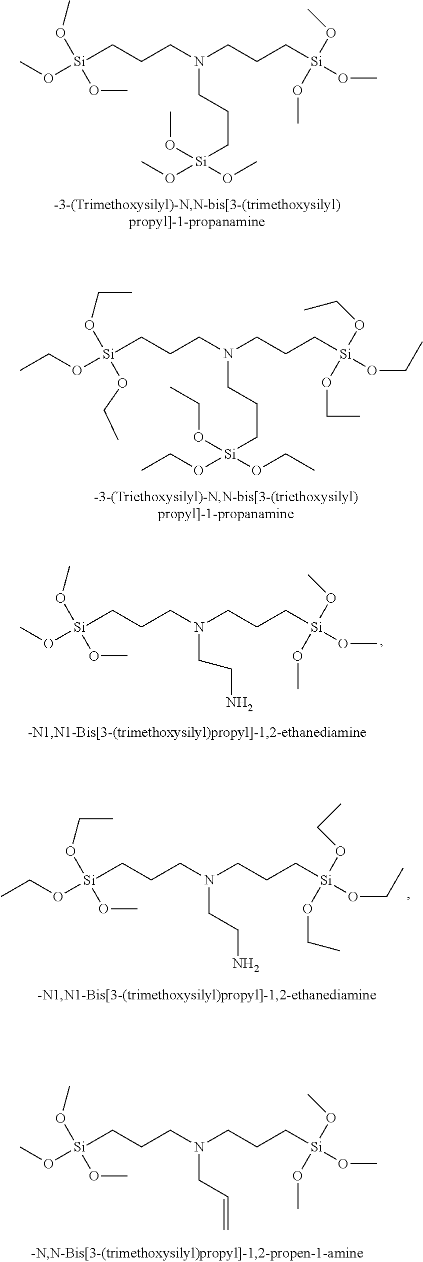

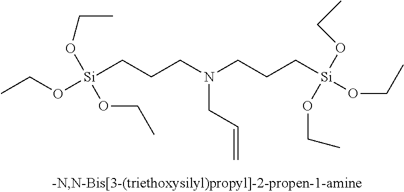 [SMILES: C=CCN(CCC[Si](OCC)(OCC)OCC)CCC[Si](OCC)(OCC)OCC]

The aforementioned organic silicon compounds of formula (II) are commercially available.

Bis(trimethoxysilylpropyl)amines with the CAS number 82985-35-1 can be purchased from Sigma-Aldrich.

Bis[3-(triethoxysilyl)propyl]amine, also known as 3-(triethoxysilyl)-N-[3-(triethoxysilyl)propyl]-1-propanamine, having the CAS number 13497-18-2, can be purchased, for example, from Sigma-Aldrich or is commercially available under the product name Dynasylan 1122 from Evonik.

N-methyl-3-(trimethoxysilyl)-N-[3-(trimethoxysilyl)propyl]-1-propanamine is alternatively referred to as Bis(3-trimethoxysilylpropyl)-N-methylamine and can be purchased commercially from Sigma-Aldrich or Fluorochem.

3-(triethoxysilyl)-N,N-bis[3-(triethoxysilyl)propyl]-1-propanamine with the CAS number 18784-74-2 can be purchased for example from Fluorochem or Sigma-Aldrich.

It has also been found to be advantageous if the agent for treating a keratinous material applied to the hair contains at least one organic silicon compound of formula (IV)

R₉Si(OR₁₀)ₖ(R₁₁)ₘ (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be referred to as silanes of the alkylalkoxysilane or alkylhydroxysilane type,

R₉Si(OR₁₀)ₖ(R₁₁)ₘ (IV).

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In a further preferred embodiment, the agent for treating a keratin material contains, in addition to the organic silicon compounds of formula (I), at least one further organic silicon compound of formula (IV)

R₉Si(OR₁₀)ₖ(R₁₁)ₘ (IV).

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In a likewise preferred embodiment, the agent for treating a keratin material contains at least one further organic silicon compound of formula (IV) in addition to the organic silicon compounds of formula (II)

R₉Si(OR₁₀)ₖ(R₁₁)ₘ (IV).

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In a further preferred embodiment, the agent for treating a keratin material contains, in addition to the organic silicon compounds of formula (I) and (II), at least one further organic silicon compound of formula (IV)

R₉Si(OR₁₀)ₖ(R₁₁)ₘ (IV).

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In the organic silicon compounds of formula (IV), the radical R₉ represents a C₁-C₁₂ alkyl group. This C₁-C₁₂ alkyl group is saturated and can be linear or branched. Preferably R9 stands for a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferably, R₉ represents a methyl group, an ethyl group or an n-octyl group.

In the organic silicon compounds of formula (IV), the radical R₁₀ represents a hydrogen atom or a C₁-C₆ alkyl group. It is particularly preferred that R₁₀ represents a methyl group or an ethyl group.

In the organic silicon compounds of formula (IV), the radical R₁₁ represents a C₁-C₆ alkyl group. It is particularly preferred that R₁₁ represents a methyl group or an ethyl group.

Furthermore k stands for a whole number from 1 to 3, and m stands for the whole number 3-k. If k stands for the number 3, then m is equal to 0. If k stands for the number 2, then m is equal to 1. If k stands for the number 1, then m is equal to 2.

A very high “anti-pollution” effect could be obtained if the agent for treating a keratinous material contains at least one organic silicon compound of formula (IV) in which the radical k represents the number 3. In this case the rest m stands for the number 0.

Organic silicon compounds of the formula (IV) that are particularly suitable for solving the problem are

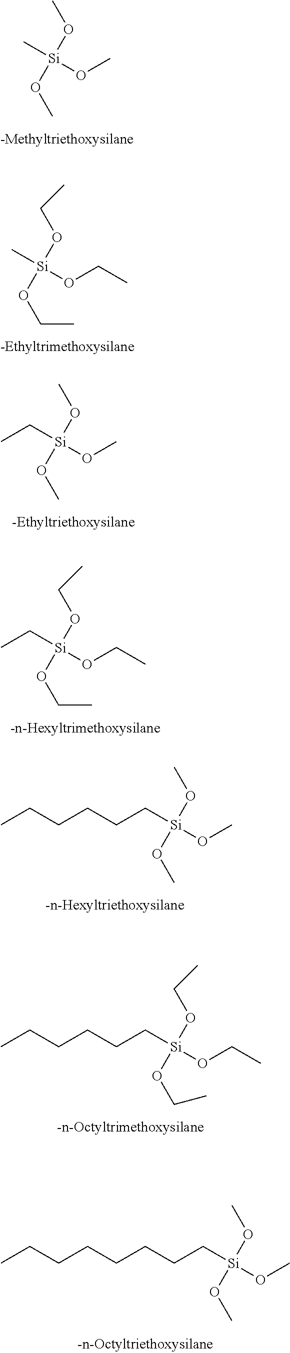

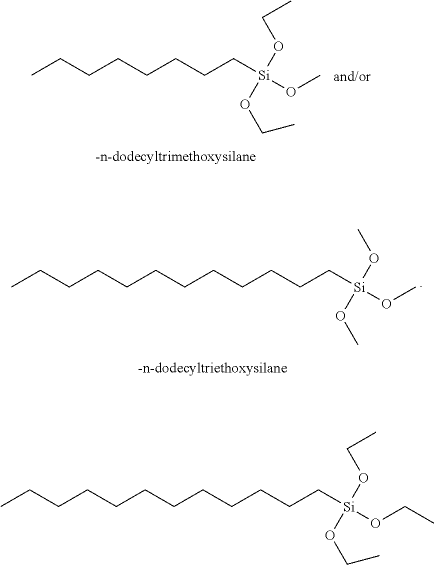

and also, propyltrimethoxysilane, propyltriethoxysilane, octadecyltrimethoxysilane and/or octadecyltriethoxysilane.

The organic silicon compounds described above are reactive compounds.

It was found that particularly stable and uniform films could be obtained on the keratinous material even when the agent contained two structurally different organic silicon compounds.

In a preferred embodiment, an agent exemplified comprises at least one organic silicone compound of formula (I) and at least one organic silicone compound of formula (IV).

In an explicitly very particularly preferred embodiment, an agent contains at least one organic silicone compound of formula (I) selected from the group consisting of (3-aminopropyl)triethoxysilane and (3-aminopropyl)trimethoxysilane, and additionally containing at least one organic silicone compound of formula (IV) selected from the group consisting of methyltrimethoxysilane, methyltriethoxysilane, ethyltrimethoxysilane, ethyltriethoxysilane, propyltrimethoxysilane, propyltriethoxysilane, hexyltrimethoxysilane and hexyltriethoxysilane.

In another preferred embodiment, an agent contains, based on the total weight of the agent-:

From about 0.5 to about 5 weight % of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3 -dimethylaminopropyl)triethoxysilane (2-dimethylaminoethyl)trimethoxysilane and (2-dimethylaminoethyl)triethoxysilane, and

From about 3.2 to about 10 wt. % of at least one second organic silicon compound selected from the group including methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

Even the addition of small amounts of water leads to hydrolysis in the case of organic silicon compounds having at least one hydrolyzable group. The hydrolysis products and/or organic silicon compounds having at least one hydroxyl group can react with one another in a condensation reaction. For this reason, both the organosilicon compounds having at least one hydrolyzable group and their hydrolysis and/or condensation products may be present in the composition. When organosilicon compounds having at least one hydroxyl group are used, both the organic silicon compounds having at least one hydroxyl group and their condensation products may be present in the composition.

A condensation product is understood to be a product formed by the reaction of at least two organic silicon compounds each having at least one hydroxyl group or hydrolyzable group per molecule with elimination of water and/or with elimination of an alkanol. The condensation products can be, for example, dimers, but also trimers or oligomers, with the condensation products being in equilibrium with the monomers. Depending on the amount of water used or consumed in the hydrolysis, the equilibrium of monomeric organic silicon compounds shifts to condensation product.

In the context of the present invention, figures in wt.-% are—unless otherwise stated—always based on the total weight of the cosmetic agent.

As a second ingredient essential to the invention, the cosmetic agent for treating a keratinous material comprises a polycarboxylic acid composed of at least one first monomer of formula (A)

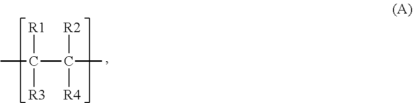

located in the
R1 and R3 independently of one another represent H, CH₃, OH, (CH₂)ₙOH, NH₂, (CH₂)NH₂, COOH, (CH₂)ₙCOOH, COOX, (CH₂)ₙCOOX, in particular H, and n is from 0 to 6 or from 1 to 6,
R2 represents H, CH₃, OH, (CH₂)nOH, NH₂, (CH₂)NH₂, COOH, (CH₂)ₙCOOH, COOX, (CH₂)ₙCOOX with n equal to 0 to 6 or 1 to 6, in particular H, CH₃, COOH or COOX, and
R4 represents (CH₂)ₙCOOH or (CH₂)nCOOX with n equal to 0 to 6 or 1 to 6, in particular 1,
wherein X in R1, R2, R3, R4 represents an alkali or alkaline earth metal ion or an ammonium ion. In the course of the work leading to the present invention, it has been found that in order to achieve advantageous properties with regard to the shaping of the hair while at the same time providing structural protection against the effects of heat, it is advantageous if the organic silicon compounds, in particular 3-(triethoxysilyl)-N-[3-(triethoxysilyl)propyl]-1-propanamine, i.e. a bis(triethoxysilylpropyl)amine, and/or (3-aminopropyl)triethoxysilane, i.e. an aminopropyltriethoxysilane (AMEO), is combined with the polycarboxylic acid.

Due to the substituent R4, the polymer composed of at least one unit according to formula (A) is a polycarboxylic acid. In the case where R1 and R3 represent hydrogen, R4 represents a carboxylic acid group, and R2 represents hydrogen or a methyl group, the polycarboxylic acid is polyacrylic acid or polymethacrylic acid. In the case where R1 and R3 represent hydrogen, R2 represents a carboxylic acid group, and R4 represents a (CH₂)COOH group, the polycarboxylic acid is itaconic acid.

Homopolyacrylic acid (INCI: Carbomer), which is commercially available in various forms under the name Carbopol®, is suitable as a strengthening compound and constitutes a polycarboxylic acid within the meaning of the present invention.

In principle, the polycarboxylic acid should be a homo- or copolymer. In the case of a copolymer, this can be composed of two, three or four different monomers. The acid group(s) of the polycarboxylic acid may be neutralized, partially neutralized or present as a salt of the carboxylic acid. In the case of the salt, the counterion can in principle be any physiologically compatible counterion, preferably comprising an alkali metal or alkaline earth metal ion or comprising an ammonium ion.

The combination of the Bis(triethoxysilylpropyl)amine and/or the aminopropyltriethoxysilane with the polycarboxylic acid forms a layer on the hair. This ensures that the hair can be well shaped temporarily and a good hairstyle hold is achieved. Furthermore, the hair surface is re-hydrophobized in oxidatively damaged hair, which leads to the reduction of frizz. In addition, structural protection against the effects of heat is improved.

In order to meet the different requirements for agents for the treatment of a keratinous material in the form of an agent for the temporary reshaping of a keratinous material (=styling agent), a large number of synthetic polymers have already been developed as strengthening compounds which can be used in an agent for the treatment of a keratinous material. It has been found that the polycarboxylic acid used in the cosmetic agent according to claim 1 is well suited, in combination with the organic silicon compounds, to sufficiently solve the problem underlying the invention.

According to a preferred embodiment of the present invention, the amount of polycarboxylic acid is from about 0.1 to about 10% by weight, preferably from about 0.5 to about 8% by weight, more preferably from about 1 to about 6% by weight, based on the total weight of the cosmetic agent, in the cosmetic agent, wherein the calculation of the amount is carried out on the basis of the free acid, regardless of whether the polycarboxylic acid is present as free acid or (partially) neutralized.

According to a preferred embodiment of the present invention, the polycarboxylic acid is a homopolymer built up from itaconic acid or a salt of itaconic acid, or the polycarboxylic acid is a copolymer of formula (B):

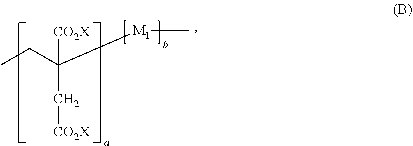

in which M₁ represents at least one or more different repeating units representing one or more different vinyl monomer units, in which X represents hydrogen or an alkali metal or alkaline earth metal, a is the molar fraction of the itaconic acid-derived repeating unit in the copolymer, and b is the molar fraction of the one or more different repeating units M₁ in the copolymer, where a is from 50 mol % to less than 100 mol %, preferably from about 20 mol % to less than 100 mol %, and b is from more than 0 mol % to 50 mol %, preferably from more than 0 mol % to 80 mol %.

Either the polycarboxylic acid of this preferred embodiment is a polyitaconic acid homopolymer, or a copolymer in which the monomer unit is derived from another monomer M₁ in addition to the itaconic acid unit. In a further embodiment, the monomer unit M₁ may also comprise two different monomers. The molar unit b is then the sum of the molar units of the various monomers. An example of such a polymer is PVP/VA/Itaconic Acid Copolymer (INCI).

The polycarboxylic acid acts as a film former in the cosmetic agent. This is deposited on the hair and accounts for the temporary deformability of the hair. As contemplated herein, the film former is a homo- or copolymer derived from itaconic acid. If the film former is present exclusively in the form of polymerized itaconic acid and/or a salt of itaconic acid, the film former forms a homopolymer.

The technical production of itaconic acid is conventionally carried out biotechnologically by submerged fermentation of molasses and other substrates using strains of certain fungi. Thus, the monomer used to produce the film former represents a raw material that is not fully synthetic and does not come directly from petrochemical sources. The advantage of using polymers of itaconic acid as film formers also lies in the conservation of resources.

It may be advantageous for the agent for treating a keratinous material to contain, in addition to the polycarboxylic acid, one or more further firming compounds, one of which is the polycarboxylic acid necessarily contained in the cosmetic agent. Furthermore, other strengthening compounds, preferably selected from the group consisting of waxes and/or other synthetic polymers may be included in the cosmetic agent. In other words, waxes and/or other synthetic polymers can be used as strengthening compounds. Ideally, the polysaccharides and the other strengthening compounds result in a film when applied to the keratin material, which on the one hand gives the hairstyle a strong hold, but on the other hand is sufficiently flexible not to break under stress.

The other synthetic polymers can be divided into cationic, anionic, nonionic and amphoteric strengthening polymers.

Suitable synthetic polymers include, for example, polymers with the following INCI names: Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/Hydroxyesters Acrylates Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquatemium-11, Polyquaternium-12, Polyquaternium-13, Polyquatemium-14, Polyquatemium-15, Polyquaternium-16, Polyquaternium-17, Polyquatemium-18, Polyquatemium-19, Polyquaternium-20, Polyquaternium-22, Polyquaternium-24, Polyquaternium-27, Polyquaternium-28, Polyquaternium-29, Polyquaternium-30, Polyquatemium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer. Cellulose ethers such as hydroxypropyl cellulose, hydroxyethyl cellulose and methyl hydroxypropyl cellulose are also suitable.

Preferably, the fixing compound is a polymer containing vinylpyrrolidone. Particularly preferably, the firming compound comprises a polymer selected from the group including polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), and mixtures thereof.

Accordingly, it is particularly preferred that the firming compound comprises a synthetic polymer selected from the group including polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), octylacrylamide/acrylates/butylaminoethyl methacrylate copolymer (INCI), and mixtures thereof.

In addition, an octylacrylamide/acrylates/butylaminoethyl methacrylate copolymer (INCI) may be contained as a stabilizing polymer in the cosmetic product. This copolymer is marketed under the terms “Amphomer®”, “Amphomer® HC”, “Amphomer® 4961”, “Amphomer® EDGE” and “Amphomer® LV 71” in various variations by Akzo Nobel.

The agent for treating a keratinous material may comprise, in particular, an agent for temporarily reshaping a keratinous material, an agent for cleaning a keratinous material, an agent for caring for a keratinous material, and/or an agent for caring for and cleaning a keratinous material.

Further ingredients of the hair treatment compositions are described below, which may be included in the compositions in addition to the mandatory and optional ingredients described above.

It may be preferred that the agent for treating a keratinous material further comprises from about 0.001 to about 20% by weight of at least one quaternary compound. This applies in particular to agents which additionally give care properties to the keratin material.

It is preferred that the at least one quaternary compound is selected from at least one of the groups consisting of
i) the monoalkylquats;
ii) the esterquats; and
iii) the quaternary imidazolines of formula (Tkat2),

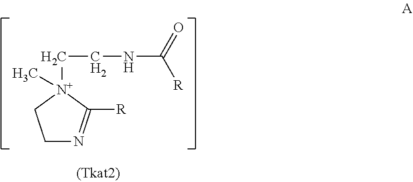

in which the radicals R independently of one another each represent a saturated or unsaturated, linear or branched hydrocarbon radical having a chain length of 8 to 30 carbon atoms and A represents a physiologically tolerated anion, and/or
iv) of the amidoamines and/or cationized amidoamines and/or
v) Poly(methacryloyloxyethyltrimethylammonium compounds) and/or;
vi) quaternized cellulose derivatives, in particular polyquaternium 10, polyquaternium-24, polyquaternium-27, polyquaternium-67, polyquaternium-72, and/or
vii) cationic alkyl polyglycosides and/or
viii) cationized honey and/or
ix) cationic guar derivatives and/or
x) chitosan and/or
xi) quaternized polyvinyl alcohol,
and mixtures thereof.

The cosmetic compound may contain, in addition or as an alternative to a synthetic polymer, at least one natural or synthetic wax having a melting point above 37° C. as a firming compound.

Natural or synthetic waxes may be solid kerosenes or isoparaffins, vegetable waxes such as candelilla wax, carnauba wax, esparto grass wax, Japan wax, cork wax, sugar cane wax, ouricury wax, montan wax, sunflower wax, fruit waxes and animal waxes such as beeswaxes and other insect waxes, Whale wax, shellac wax, wool wax and brushing grease, furthermore mineral waxes, such as ceresin and ozokerite or petrochemical waxes, such as petrolatum, kerosene waxes, microwaxes of polyethylene or polypropylene and polyethylene glycol waxes can be used. It may be advantageous to use hydrogenated or cured waxes. Chemically modified waxes, in particular hard waxes such as montan ester waxes, sasol waxes and hydrogenated jojoba waxes, can also be used.

Also suitable are the triglycerides of saturated and optionally hydroxylated C16-30 fatty acids, such as hydrogenated triglyceride fats (hydrogenated palm oil, hydrogenated coconut oil, hydrogenated castor oil), glyceryl tribehenate or glyceryl tri-12-hydroxystearate.

The wax components can also be selected from the group of esters of saturated, unbranched alkanecarboxylic acids having a chain length of 22 to 44 carbon atoms and saturated, unbranched alcohols having a chain length of 22 to 44 carbon atoms, provided that the wax component or the totality of wax components are solid at room temperature. Silicone waxes, for example stearyltrimethylsilane/stearyl alcohol, may also be beneficial.

Natural, chemically modified, and synthetic waxes can be used alone or in combination. Thus, several waxes can also be used. Furthermore, a number of wax compounds, possibly mixed with other additives, are also commercially available. The products sold under the designations “Special Wax 7686 OE” (a mixture of cetyl palmitate, beeswax, microcrystalline wax and polyethylene with a melting range of 73-75° C.; manufacturer: Kahl & Co), Polywax® GP 200 (a mixture of stearyl alcohol and polyethylene glycol stearate with a melting point of 47-51° C.; manufacturer: Croda) and “Softceresin® FL 400” (a vaseline/vaseline oil/wax mixture with a melting point of 50-54° C.; manufacturer: Parafluid Mineral Oil Company) are examples of mixtures that can be used.

Preferably, the wax is selected from carnauba wax (INCI: Copernicia Cerifera Cera) Beeswax (INCI: Beeswax), petrolatum (INCI), microcrystalline wax and especially mixtures thereof.

Preferred blends include the combination of carnauba wax (INCI: Copernicia Cerifera Cera), petrolatum and microcrystalline wax or the combination of beeswax (INCI: Beeswax) and Petrolatum.

Wax or the wax components should be solid at 25° C. and should melt in the range >37° C.

The composition for treating a keratinous material contains the strengthening compound, if it does not concern the polycarboxylic acid used obligatorily, preferably in a total amount of about 0.5 to about 50% by weight, preferably from about 1 to about 40% by weight, more preferably from about 1.5 to about 30% by weight, still more preferably from about 2 to about 25% by weight, relative to the total weight of the cosmetic composition.

Other suitable ingredients include nonionic polymers, anionic polymers, (further) cationic polymers, waxes, protein hydrolysates, amino acids, oligopetides, vitamins, provitamins, vitamin precursors, betaines, biochinones, purine (derivatives), care substances, plant extracts, silicones, ester oils, UV light filters, thickeners, electrolytes, pH adjusters, swelling agents, colorants, anti-dandruff agents, complexing agents, opacifiers, pearlescent agents, pigments, stabilizers, propellants, antioxidants, perfume oils and/or preservatives.

In preferred embodiments 1 to 94, the organic silicon compounds of the following table are combined with polyitaconic acid or PVP/VA/Itaconic Acid Copolymer (INCI) in a cosmetic agent. Here, the polyitaconic acid is either a homopolymer or a copolymer comprising a monomer unit derived from itaconic acid. The following combinations are present in cosmetic products in preferred embodiments of the present disclosure with other ingredients described above:

| | Silane compound | other ingredients |
|---|---|---|
| 1 | (3-Aminopropyl)trimethoxysilane | Polyitaconic acid |
| 2 | (3-Aminopropyl)triethoxysilan | Polyitaconic acid |
| 3 | (2-Aminoethyl)trimethoxysilane | Polyitaconic acid |
| 4 | (2-Aminoethyl)triethoxysilane | Polyitaconic acid |
| 5 | (3-Dimethylaminopropyl)trimethoxysilane | Polyitaconic acid |
| 6 | (3-Dimethylaminopropyl)triethoxysilane | Polyitaconic acid |
| 7 | (2-Dimethylaminoethyl)trimethoxysilane | Polyitaconic acid |
| 8 | (2-Dimethylaminoethyl)triethoxysilane | Polyitaconic acid |
| 9 | 3-(trimethoxysilyl)-N-[3-(trimethoxysilyl)propyl]-1- | Polyitaconic acid |
| | propanamine | |
| 10 | 3-(triethoxysilyl)-N-[3-(triethoxysilyl)propyl]-1- | Polyitaconic acid |
| | propanamine | |
| 11 | N-methyl-3-(trimethoxysilyl)-N-[3- | Polyitaconic acid |
| | (trimethoxysilyl)propyl]-1-propanamine | |
| 12 | N-Methyl-3-(triethoxysilyl)-N-[3- | Polyitaconic acid |
| | (triethoxysilyl)propyl]-1-propanamine | |
| 13 | 2-[Bis[3-(trimethoxysilyl)propyl]amino]-ethanol | Polyitaconic acid |
| 14 | 2-[bis[3-(triethoxysilyl)propyl]amino]ethanol | Polyitaconic acid |
| 15 | 3-(trimethoxysilyl)-N,N-bis[3-(trimethoxysilyl)propyl]- | Polyitaconic acid |
| | 1-propanamine | |
| 16 | 3-(triethoxysilyl)-N,N-bis[3-(triethoxysilyl)propyl]-1- | Polyitaconic acid |
| | propanamine | |
| 17 | N1,N1-Bis[3-(trimethoxysilyepropyl]-1,2- | Polyitaconic acid |
| | ethanediamine | |
| 18 | N1,N1-Bis[3-(triethoxysilyl)propyl]-1,2-ethanediamine | Polyitaconic acid |
| 19 | N,N-Bis[3-(trimethoxysilyl)propyl]-2-propen-1-amine | Polyitaconic acid |
| 20 | N,N-Bis[3-(triethoxysilyl)propyl]-2-propen-1-amine | Polyitaconic acid |
| 21 | Methyltrimethoxysilane | Polyitaconic acid |
| 22 | Methyltriethoxysilane | Polyitaconic acid |
| 23 | Ethyltrimethoxysilane | Polyitaconic acid |
| 24 | Ethyltriethoxysilane | Polyitaconic acid |
| 25 | Octyltrimethoxysilane | Polyitaconic acid |
| 26 | Octyltriethoxysilane | Polyitaconic acid |
| 27 | Dodecyltrimethoxysilane | Polyitaconic acid |
| 28 | Dodecyltriethoxysilane | Polyitaconic acid |
| 29 | (3-Aminopropyl)trimethoxysilane | PVP/VA/Itaconic Acid |
| | | Copolymer (INCI) |
| 30 | (3-Aminopropyl)triethoxysilan | PVP/VA/Itaconic Acid |
| | | Copolymer (INCI) |
| 31 | (2-Aminoethyl)trimethoxysilane | PVP/VA/Itaconic Acid |
| | | Copolymer (INCI) |
| 32 | (2-Aminoethyl)triethoxysilane | PVP/VA/Itaconic Acid |
| | | Copolymer (INCI) |
| 33 | (3-Dimethylaminopropyl)trimethoxysilane | PVP/VA/Itaconic Acid |
| | | Copolymer (INCI) |
| 34 | (3-Dimethylaminopropyl)triethoxysilane | PVP/VA/Itaconic Acid |
| | | Copolymer (INCI) |
| 35 | (2-Dimethylaminoethyl)trimethoxysilane | PVP/VA/Itaconic Acid |
| | | Copolymer (INCI) |
| 36 | (2-Dimethylaminoethyl)triethoxysilane | PVP/VA/Itaconic Acid |
| | | Copolymer (INCI) |
| 37 | 3-(trimethoxysilyl)-N-[3-(trimethoxysilyl)propyl]-1- | PVP/VA/Itaconic Acid |
| | propanamine | Copolymer (INCI) |
| 38 | 3-(triethoxysilyl)-N-[3-(triethoxysilyl)propyl]-1- | PVP/VA/Itaconic Acid |
| | propanamine | Copolymer (INCI) |
| 39 | N-methyl-3-(trimethoxysilyl)-N-[3- | PVP/VA/Itaconic Acid |
| | (trimethoxysilyl)propyl]-1-propanamine | Copolymer (INCI) |
| 40 | N-Methyl-3-(triethoxysilyl)-N-[3- | PVP/VA/Itaconic Acid |
| | (triethoxysilyl)propyl]-1-propanamine | Copolymer (INCI) |
| 41 | 2-[Bis [3-(trimethoxysilyl)propyl]amino]-ethanol | PVP/VA/Itaconic Acid |
| | | Copolymer (INCI) |
| 42 | 2-[bis [3-(triethoxysilyl)propyl]amino]ethanol | PVP/VA/Itaconic Acid |
| | | Copolymer (INCI) |
| 43 | 3-(trimethoxysilyl)-N,N-bis[3-(trimethoxysilyl)propyl]- | PVP/VA/Itaconic Acid |
| | 1-propanamine | Copolymer (INCI) |
| 44 | 3-(triethoxysilyl)-N,N-bis[3-(triethoxysilyepropyl]-1- | PVP/VA/Itaconic Acid |
| | propanamine | Copolymer (INCI) |
| 45 | N1,N1-Bis[3-(trimethoxysilyl)propyl]-1,2- | PVP/VA/Itaconic Acid |
| | ethanediamine | Copolymer (INCI) |
| 46 | N1,N1-Bis[3-(triethoxysilyl)propyl]-1,2-ethanediamine | PVP/VA/Itaconic Acid |
| | | Copolymer (INCI) |
| 47 | N,N-Bis[3-(trimethoxysilyl)propyl]-2-propen-1-amine | PVP/VA/Itaconic Acid |
| | | Copolymer (INCI) |
| 48 | N,N-Bis[3-(triethoxysilyepropyl]-2-propen-1-amine | PVP/VA/Itaconic Acid |
| | | Copolymer (INCI) |
| 49 | Methyltrimethoxysilane | PVP/VA/Itaconic Acid |
| | | Copolymer (INCI) |
| 50 | Methyltriethoxysilane | PVP/VA/Itaconic Acid |
| | | Copolymer (INCI) |
| 51 | Ethyltrimethoxysilane | PVP/VA/Itaconic Acid |
| | | Copolymer (INCI) |
| 52 | Ethyltriethoxysilane | PVP/VA/Itaconic Acid |
| | | Copolymer (INCI) |
| 53 | Octyltrimethoxysilane | PVP/VA/Itaconic Acid |
| | | Copolymer (INCI) |
| 54 | Octyltriethoxysilane | PVP/VA/Itaconic Acid |
| | | Copolymer (INCI) |
| 55 | Dodecyltrimethoxysilane | PVP/VA/Itaconic Acid |
| | | Copolymer (INCI) |
| 56 | Dodecyltriethoxysilane | PVP/VA/Itaconic Acid |
| | | Copolymer (INCI) |
| 57 | (3-Aminopropyl)triethoxysilan + Methyltrimethoxysilan | Polyitaconic acid |
| 58 | (3-Aminopropyl)triethoxysilane + Methyltriethoxysilane | Polyitaconic acid |
| 59 | (3-Aminopropyl)triethoxysilane + Ethyltrimethoxysilane | Polyitaconic acid |
| 60 | (3-Aminopropyl)triethoxysilane + Ethyltriethoxysilane | Polyitaconic acid |
| 61 | (3-Aminopropyl)triethoxysilane + | Polyitaconic acid |
| | Propyltrimethoxysilane | |
| 62 | (3-Aminopropyl)triethoxysilane + Propyltriethoxysilane | Polyitaconic acid |
| 63 | (3-Aminopropyl)triethoxysilane + | Polyitaconic acid |
| | Hexyltrimethoxysilane | |
| 64 | (3-Aminopropyl)triethoxysilane + Hexyltriethoxysilane | Polyitaconic acid |
| 65 | (3-Aminopropyl)triethoxysilane + Octyltrimethoxysilane | Polyitaconic acid |
| 66 | (3-Aminopropyl)triethoxysilane + Octyltriethoxysilane | Polyitaconic acid |
| 67 | (3-Aminopropyl)triethoxysilane + Dodecyltrimethoxysilane | Polyitaconic acid |
| 68 | (3-Aminopropyl)triethoxysilane + Dodecyltriethoxysilane | Polyitaconic acid |
| 69 | (3-Aminopropyl)triethoxysilane + Octadecyltrimethoxysilane | Polyitaconic acid |
| 70 | (3-Aminopropyl)triethoxysilane + | Polyitaconic acid |
| | Octadecyltriethoxysilane | |
| 71 | (3-Aminopropyl)triethoxysilan + Methyltrimethoxysilan | PVP/VA/Itaconic Acid |
| | | Copolymer (INCI) |
| 72 | (3-Aminopropyl)triethoxysilane + Methyltriethoxysilane | PVP/VA/Itaconic Acid |
| | | Copolymer (INCI) |
| 73 | (3-Aminopropyl)triethoxysilane + Ethyltrimethoxysilane | PVP/VA/Itaconic Acid |
| | | Copolymer (INCI) |
| 74 | (3-Aminopropyl)triethoxysilane + Ethyltriethoxysilane | PVP/VA/Itaconic Acid |
| | | Copolymer (INCI) |
| 75 | (3-Aminopropyl)triethoxysilane + | PVP/VA/Itaconic Acid |
| | Propyltrimethoxysilane | Copolymer (INCI) |
| 76 | (3-Aminopropyl)triethoxysilane + Propyltriethoxysilane | PVP/VA/Itaconic Acid |
| | | Copolymer (INCI) |
| 77 | (3-Aminopropyl)triethoxysilane + | PVP/VA/Itaconic Acid |
| | Hexyltrimethoxysilane | Copolymer (INCI) |
| 78 | (3-Aminopropyl)triethoxysilane + Hexyltriethoxysilane | PVP/VA/Itaconic Acid |
| | | Copolymer (INCI) |
| 79 | (3-Aminopropyl)triethoxysilane + Octyltrimethoxysilane | PVP/VA/Itaconic Acid |
| | | Copolymer (INCI) |
| 80 | (3-Aminopropyl)triethoxysilane + Octyltriethoxysilane | PVP/VA/Itaconic Acid |
| | | Copolymer (INCI) |
| 81 | (3-Aminopropyl)triethoxysilane + Dodecyltrimethoxysilane | PVP/VA/Itaconic Acid |
| | | Copolymer (INCI) |
| 82 | (3-Aminopropyl)triethoxysilane + Dodecyltriethoxysilane | PVP/VA/Itaconic Acid |
| | | Copolymer (INCI) |
| 83 | (3-Aminopropyl)triethoxysilane + Octadecyltrimethoxysilane | PVP/VA/Itaconic Acid |
| | | Copolymer (INCI) |
| 84 | (3-Aminopropyl)triethoxysilane + Octadecyltriethoxysilane | PVP/VA/Itaconic Acid |
| | | Copolymer (INCI) |
| 85 | Propyltrimethoxysilane | Polyitaconic acid |
| 86 | Propyltriethoxysilane | Polyitaconic acid |
| 85 | Propyltrimethoxysilane | PVP/VA/Itaconic Acid |
| | | Copolymer (INCI) |
| 86 | Propyltriethoxysilane | PVP/VA/Itaconic Acid |
| | | Copolymer (INCI) |
| 87 | Hexyltrimethoxysilane | Polyitaconic acid |
| 88 | Hexyltriethoxysilane | Polyitaconic acid |
| 89 | Hexyltrimethoxysilane | PVP/VA/Itaconic Acid |
| | | Copolymer (INCI) |
| 90 | Hexyltriethoxysilane | PVP/VA/Itaconic Acid |
| | | Copolymer (INCI) |
| 91 | Octadecyltrimethoxysilane | Polyitaconic acid |
| 92 | Octadecyltriethoxysilane | Polyitaconic acid |
| 93 | Octadecyltrimethoxysilane | PVP/VA/Itaconic Acid |
| | | Copolymer (INCI) |
| 94 | Octadecyltriethoxysilane | PVP/VA/Itaconic Acid |
| | | Copolymer (INCI) |

The active ingredient combination of at least one organic silicon compound and the polycarboxylic acid may already be present in the agent for treating a keratinous material. In this embodiment, the agent for treating a keratinous material is already distributed in a ready-to-use form. In order to provide a formulation that is as stable as possible during storage, the agent itself is preferably packaged with low or no water.

Alternatively, at least one organic silicon compound is added a maximum of 12 hours, preferably a maximum of 6 hours, more preferably a maximum of 3 hours, even more preferably a maximum of 1 hour prior to application of the keratinous material treatment agent to a base comprising all of the ingredients of the keratinous material treatment agent except the at least one organic silicon compound.

Furthermore, the organosilicon compound, in particular the (3-aminopropyl)triethoxysilane, and the polycarboxylic acid are alternatively added to a cosmetic product only shortly before use, i.e. 1 minute to 12 hours, preferably from about 2 minutes to about 6 hours, particularly preferably from about 1 minute to about 3 hours, especially preferably from about 1 minute to about 1 hour.

In another alternative, the organosilicon compound, in particular the (3-aminopropyl)triethoxysilane, is added to an aqueous solution which is applied to the hair and, in the second step, an aqueous solution or cosmetic agent containing the polycarboxylic acid is applied to the hair.

For example, the user may first mix or shake an agent (α) comprising the organic silicon compound(s) with an agent (β) comprising the remaining ingredients of the agent for treating a keratinous material. The user can now apply this mixture of (α) and (β)—either directly after its preparation or after a short reaction time of 1 minute to 20 minutes—to the keratinous materials. The agent (β) may contain water, in particular water in an amount >30% by weight, based on the total weight of the agent for treating keratinous materials.

Another subject matter of the present application is the use of a cosmetic agent as contemplated herein for treating a keratinous material
for shaping keratinous material,
for the care of keratinous material.
to reduce and/or prevent harmful effects of air and water contaminants on keratinous material and/or
to reduce and/or prevent the harmful effects of heat exposure on a keratinous material.

With regard to further preferred embodiments of use, the same applies mutatis mutandis as to the cosmetic agents.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic agent for the treatment of a keratinous material, comprising
a) at least one organic silicon compound and
b) at least one polycarboxylic acid which is composed of at least one first monomer of the formula (A) 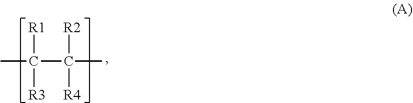
wherein
R1 and R3 independently of one another represent H, CH₃, OH, (CH₂)ₙOH, NH₂, (CH₂)NH₂, COOH, (CH₂)ₙCOOH, COOX, (CH₂)ₙCOOX, in particular H, and n is from 0 to 6 or from 1 to 6,
R2 represents H, CH₃, OH, (CH₂)nOH, NH₂, (CH₂)NH₂, COOH, (CH₂)ₙCOOH, COOX, (CH₂)ₙCOOX with n equal to from 0 to 6 or from 1 to 6, in particular H, CH₃, COOH or COOX, and
R4 represents (CH₂)ₙCOOH or (CH₂)nCOOX with n equal to 0 to 6 or from 1 to 6,
wherein X in R1, R2, R3, R4 represents an alkali or alkaline earth metal ion or an ammonium ion.

2. A cosmetic composition for treating a keratinous material according to claim 1,
wherein
the at least one organic silicon compound comprises a compound of the formula (I) or of formula (II),
wherein in the organic silicon compound of formula (I)
R₁R₂N-L-Si(OR₃)ₐ(R₄)_{b} (I),
R₁, R₂ both represent a hydrogen atom,
L represents a linear, divalent C₁-C₆-alkylene group, preferably a propylene group (—CH₂—CH₂—CH₂—) or an ethylene group (—CH₂—CH₂—),
R₃, R₄ independently represent a methyl group or an ethyl group,
a stands for the number 3 and
b stands for the number 0, and
wherein in the organic silicon compound of formula (II)
(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
R₅, R_{5′}, R_{5″}, R₆, R_{6′} and R_{6″} stand independently for a C₁-C₆ alkyl group,
A, A′, A″, A″′ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)
-(A″″)-Si(R₆″)_{d}″(OR₅″)_{c}″ (III),
c stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
with the proviso that at least one of the components of e, f, g and h is different from 0.

3. A cosmetic composition for treating a keratinous material according to claim 1,
wherein
agent for treating a keratinous material contains at least one organic silicon compound of formula (I) selected from the group consisting of
(3-Aminopropyl)trimethoxysilane
(3-Aminopropyl)triethoxysilane
(2-Aminoethyl)trimethoxysilane
(2-Aminoethyl)triethoxysilane
(3-Dimethylaminopropyl)trimethoxysilane
(3-Dimethylaminopropyl)triethoxysilane
(2-dimethylaminoethyl)trimethoxysilane, and
(2-Dimethylaminoethyl)triethoxysilane,
and wherein,
the agent for treating a keratinous material contains at least one organic silicon compound of formula (II) selected from the group consisting of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-Methyl-3-(triethoxysilyl)-N-[3-(triethoxysilyl)propyl]-1-propanamine
2-[Bis[3-(trimethoxysilyl)propyl]amino]-ethanol
2-[Bis[3-(triethoxysilyl)propyl]amino]ethanol
3-(Trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-Bis[3-(trimethoxysilyl)propyl]-2-propen-1-amine and
N,N-Bis[3-(triethoxysilyl)propyl]-2-propen-1-amine.

4. Cosmetic agent for the treatment of a keratinous material according to claim 1, wherein the organic silicon compound is present in an amount of from about 0.01 to about 10% by weight based on the total weight of the cosmetic agent.

5. The cosmetic agent for treating a keratinous material according to claim 1, wherein the polycarboxylic acid is a homopolymer composed of itaconic acid or a salt of itaconic acid, or wherein the polycarboxylic acid is a copolymer of formula (B): 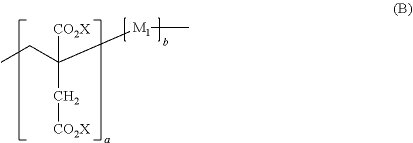
in which M₁ represents at least one or more different repeating units representing one or more different vinyl monomer units, in which X represents hydrogen or an alkali metal or alkaline earth metal, a is the molar fraction of the itaconic acid-derived repeating unit in the copolymer, and b is the molar fraction of the one or more different repeating units M₁ in the copolymer, where a is from about 20 mol % to less than 100 mol %.

6. A cosmetic agent for treating a keratinous material according to claim 1, wherein the polycarboxylic acid is present in the cosmetic agent in an amount of from about 0.1 to about 10% by weight based on the total weight of the cosmetic agent.

7. Cosmetic agent for treating a keratinous material according to claim 1, wherein the agent for treating a keratinous material comprises at least one organic silicon compound of formula (IV),
R₉Si(OR₁₀)ₖ(R₁₁)ₘ (IV),
where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

8. Cosmetic agent for treating a keratinous material according to claim 1, wherein the agent for treating a keratinous material comprises at least two structurally different organic silicon compounds.

9. A cosmetic agent for treating a keratinous material according to claim 1, wherein the agent for treating a keratinous material comprises—based on the total weight of the agent for treating a keratinous material:
from about 0.5 to about 3 weight % of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
from about 3.2 to about 7 wt. % of at least one second organic silicon compound selected from the group of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

10. (canceled)

11. The cosmetic agent according to claim 4, wherein the organic silicon compound comprises 3-(triethoxysilyl)-N-[3-(triethoxysilyl)propyl]-1-propanamine or (3-aminopropyl)triethoxysilane.

12. The cosmetic agent according to claim 5, wherein a is greater than 50 mole %.

13. The cosmetic agent according to claim 7, wherein the organic silicon compound comprises a compound selected from the group consisting of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane, and octadecyltriethoxysilane.

14. The cosmetic agent according to claim 1, wherein the polycarboxylic acid comprises a homopolymer or copolymer of itaconic acid or a salt thereof.

15. The cosmetic agent according to claim 5, wherein the polycarboxylic acid comprises a copolymer of vinylpyrrolidone, vinyl acetate, and itaconic acid.

16. A method of treating keratinous material, comprising applying a cosmetic agent according to claim 1 onto the keratinous material.

17. The method according to claim 16, comprising applying an aqueous solution comprising the organic silicon compound onto the keratinous material and applying an aqueous solution comprising the polycarboxylic acid onto the keratinous material.

18. The method according to claim 16, comprising mixing an agent (α) comprising the organic silicon compound(s) with an agent (β) comprising the remaining ingredients of the agent and apply the mixture of (α) and (β) to the keratinous material directly after its preparation or after a suitable reaction time.

19. The method according to claim 16, wherein the polycarboxylic acid comprises a homopolymer or a copolymer of itaconic acid or a salt thereof.
